(19) United States
(12) Patent Application Publication
(10) Pub. No.: US 2018/0371539 A1
(43) Pub. Date: Dec. 27, 2018

(54) Method of detecting a pre-determined event in a nucleic acid sample and system thereof

(71) Applicant: BGI Diagnosis Co., Ltd., Shenzhen (CN)

(72) Inventors: Hui Jiang, Shenzhen (CN); Fang Chen, Shenzhen (CN); Huijuan Ge, Shenzhen (CN); Peipei Li, Shenzhen (CN); Xuchao Li, Shenzhen (CN); Jian Wang, Shenzhen (CN); Jun Wang, Shenzhen (CN); Huanming Yang, Shenzhen (CN)

(21) Appl. No.: 16/023,868

(22) Filed: Jun. 29, 2018

Related U.S. Application Data
(63) Continuation of application No. 14/351,468, filed on Apr. 11, 2014.

(30) Foreign Application Priority Data
Oct. 14, 2011 (CN) 201110311333.2

(51) Int. Cl. C12Q 1/6874

(52) U.S. Cl. CPC C12Q 1/6874; C12Q 2600/156; C12Q 1/6869

(57) ABSTRACT

Disclosed are a method of detecting a pre-determined event in a nucleic acid sample and a system thereof. The method of detecting the pre-determined event in the nucleic acid sample comprises the following steps: constructing a sequencing-library for the nucleic acid sample; sequencing the sequencing-library to obtain a sequencing result consisting of a plurality of sequencing data; determining the sequencing data from a pre-determined region; and determining an occurrence of the pre-determined event in the nucleic acid sample based on a composition of the sequencing data from the pre-determined region.

### CROSS-REFERENCE TO RELATED APPLICATION

This application is a continuation of U.S. patent application Ser. No. 14/351,468, filed Apr. 11, 2014, which is a Section 371 National Stage Application of International Application No. PCT/CN/2011/084380, filed Dec. 21, 2011, and published as WO2013/053182 on Apr. 18, 2013, which claims priority to and benefits of Chinese Patent Application Serial No. 201110311333.2, filed with the State Intellectual Property Office of P. R. China on Oct. 14, 2011, the entire contents of which are incorporated herein by reference.

### FIELD

The present disclosure relates to biomedicine field, and more particularly to method, system and capturing chip for detecting pre-determined event in nucleic acid.

### BACKGROUND

Monogenic disorders is a disease or pathological trait controlled by a pair of allele, also known as Mendel disease or monogenic disease, which may be classified as autosomal recessive genetic disease (AR), autosomal dominant genetic disease (AD), X-linked recessive genetic disease (XR), X-linked dominant genetic disease (XD) and Y-linked genetic disease, et al. According to a data publish on Human Genome Project Information Website, so far there are 6000 kinds of monogenic diseases having known clinic symptom and explicit genetic mechanism.

But, the current detecting method still needs to be improved.

### SUMMARY

Embodiments of the present disclosure seek to solve at least one of the problems existing in the prior art to at least some extent. Thus, one objective of the present disclosure directs to provide a method of effectively detecting a pre-determined event in a nucleic acid sample.

According to a first broad aspect of the present disclosure, there is provided a method of detecting a pre-determined event in a nucleic acid sample. According to embodiments of the present disclosure, the method of detecting the pre-determined event in the nucleic acid sample may comprise following steps: constructing a sequencing-library for the nucleic acid sample; sequencing the sequencing-library to obtain a sequencing result consisting of a plurality of sequencing data; determining the sequencing data from a pre-determined region; and determining an occurrence of the pre-determined event in the nucleic acid sample based on a composition of the sequencing data from the pre-determined region. The pre-determined event in the nucleic acid sample may be effectively detected using the above method, for example, a mutation type of a SNP site may be effectively detected using the above method, or an aneuploidy of a prenatal chromosome may be effectively detected using the above method.

According to a second broad aspect of the present disclosure, there is provided a system of detecting a pre-determined event in a nucleic acid sample. According to embodiments of the present disclosure, the system of detecting the pre-determined event in the nucleic acid sample may comprise: a library-constructing apparatus, suitable for constructing a sequencing-library for the nucleic acid sample; a sequencing apparatus, connected to the library-constructing apparatus, suitable for sequencing the sequencing-library to obtain a sequencing result consisting of a plurality of sequencing data; an analysis apparatus, suitable for determining the sequencing data from a pre-determined region and determining an occurrence of the pre-determined event in the nucleic acid sample based on a composition of the sequencing data from the pre-determined region. Using the system may effectively perform the above-mentioned method of detecting the pre-determined event in the nucleic acid sample, thereby the pre-determined event in the nucleic acid sample may be effectively detected, for example, a mutation type of a SNP site may be effectively detected, or an aneuploidy of a prenatal chromosome may be effectively detected.

According to a third broad aspect of the present disclosure, there is provided a capturing chip. According to embodiments of the present disclosure, the capturing chip may comprise: a capturing chip body; a plurality of oligonucleotide probes, configured on a surface of the capturing chip body, wherein the plurality of oligonucleotide probes are specific for the pre-determined region of human genome. The plurality of oligonucleotide probes based on the capturing chip are specific for the pre-determined region of human genome, thus, the capturing chip may be effectively applied to the above-mentioned method of detecting the pre-determined event in the nucleic acid sample, to effectively determine the sequencing data from the pre-determined region.

According to a fourth broad aspect of the present disclosure, there is provided a method of monogenic disorder diagnosis, paternity test or prenatal screening Down's syndrome. According to embodiments of the present disclosure, the method may comprise: detecting a pre-determined event in a nucleic acid sample according to the method of detecting a pre-determined event in a nucleic acid sample above-mentioned.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the accompanying drawings, in which:

FIG. 1 is a schematic diagram of a system of detecting a pre-determined event in a nucleic acid sample according to one embodiment of the present disclosure;

FIG. 2 is a schematic diagram of a system of detecting a pre-determined event in a nucleic acid sample according to another embodiment of the present disclosure;

FIG. 3 is an accuracy result of different sequencing depths obtained by calculating stimulation frequency of each base using by means of Bayesian Model shown as formula I according to one embodiment of the present disclosure, wherein the stimulation frequencies are corresponding to different sequencing depths randomly produced during SNP detection under a probability distribution of bases in the case of mother heterozygote and fetus homozygote, wherein the fetal concentration represents a percentage between fetal DNA and plasma DNA in the maternity peripheral blood, the detection efficiency represents a detection efficiency of the Model, i.e. 1-FN (false negative);

FIG. 4 is a result of detecting a chromosome aneuploidy according to one embodiment of the present disclosure; and

FIG. 5 is a schematic diagram of a capturing chip according to one embodiment of the present disclosure.

### DETAILED DESCRIPTION

Reference will be made in detail to embodiments of the present disclosure. The same or similar elements and the elements having same or similar functions are denoted by like reference numerals throughout the descriptions. The embodiments described herein with reference to drawings are explanatory, illustrative, and used to generally understand the present disclosure. The embodiments shall not be construed to limit the present disclosure. In addition, terms such as “first” and “second” are used herein for purposes of description and are not intended to indicate or imply relative importance or significance. Furthermore, in the description of the present disclosure, unless otherwise stated, the term “a plurality of” refers to two or more.

Method of Detecting a Pre-Determined Event in a Nucleic Acid Sample

According to embodiments of the present disclosure, there is provided a method of detecting a pre-determined event in a nucleic acid sample. The term “pre-determined event” used herein refers to a mutation or an abnormality which may exist in the nucleic acid sample, for example, genetic variation. An occurring site or an occurring region of the mutation or the abnormality has been already known or reported in advance, the method according to embodiments of the present disclosure, a detectable pre-determined event may be a structural variation of a nucleic sequence, for example, deletion, insertion, mutation, duplication, ectopic and inversion, etc., may also be a number variation of a chromosome, for example, an aneuploidy, etc., or may be a molecular genetic marker comprising a single nucleotide polymorphisms (SNP), microsatellite sequence (STR), etc. The inventors find out, it may effectively determine an existence of the pre-determined event or a type thereof in the nucleic acid sample, by means of detecting a specific region of the nucleic acid sample comprising a site at which the pre-determined event may occur, and analyzing a composition of sequencing data from the specific region (for example, a respective occurrence frequency of A, T, G, C base at a specific site), for example, a SNP type may be determined. It should be noted that, according to the method of the present disclosure, based on the determination of the existence of the pre-determined event, these detection results may be subjected to further analysis, which may obtain a further conclusion, for example according to embodiments of the present disclosure, after obtaining a SNP information, the method may be further applied to realize effective paternity test. Thus, the term “pre-determined event” used herein should be broadly understood, which comprises not only a data directly obtained from the sequencing result, but also a data obtained from by analyzing the sequencing result, for example, determining a genetic relationship between different nucleic acid samples.

According to embodiments of the present disclosure, the method of detecting the pre-determined event in the nucleic acid sample may comprise following steps:

Firstly, a sequencing-library is constructed for the nucleic acid sample. According to embodiments of the present disclosure, a type of the nucleic acid sample are not subjected to any special restrictions, the type of the nucleic acid sample may be deoxyribonucleic acid (DNA) or ribonucleic acid (RNA), preferably is DNA. It would be appreciated by those skilled in the art that a RNA sample may be detected by being converted to a DNA sample having a corresponding sequence by means of conventional methods. In addition, a resource of the nucleic acid sample is also not subjected to any special restrictions. According to some embodiments of the present disclosure, the nucleic acid sample may be at least one selected from a group consisting of human genomic DNA sample and free nucleic acid, preferably, the genomic DNA sample is a genomic DNA derived from human white blood cell or maternal plasma. The inventors find out that the method of the present disclosure may effectively determine the specific event in the human genome, such as nucleic acid mutation. In addition, a genetic trait of fetus may be effectively analyzed by means of analyzing human genomic DNA sample or free nucleic acid extracted from human peripheral blood, especially from maternity peripheral blood, to realize non-injured prenatal diagnosis or paternity test. A method and a process of constructing a sequencing-library for a nucleic acid sample may be selected appropriately by those skilled in the art according to different sequencing techniques. A detailed process may refer to a specification provided by sequencing-instrument manufacturer, such as Illumina Company, for example Multiplexing Sample Preparation Guide (Part#1005361; February 2010) or Paired-End SamplePrep Guide (Part#1005063; February 2010) is referred, which are both incorporated herein by reference. According to embodiments of the present disclosure, a method and an apparatus for extracting a nucleic acid sample from a biological sample are also not subjected to any special restrictions. The extraction may be performed using a commercial kit for nucleic acid extraction.

After obtained, the sequencing-library is sequenced using a sequencing apparatus to obtain a sequencing result which consists of a plurality of sequencing data. According to embodiments of the present disclosure, a method and an apparatus for performing sequencing are not subjected to any special restrictions, including but not limited to a dideoxy chain termination; a high-throughput sequencing method is preferred. Thus, the efficiency of determining an aneuploidy of nucleated red blood cell chromosome may be further improved, by utilizing the characteristics of high-throughput and deep-sequencing using the sequencing apparatus. Thereby, the accuracy and precision of subsequently analyzing the sequencing data, especially a statistical test, has been improved.

The high-throughput sequencing method includes but not limited to a Next-Generation sequencing technique or a single molecule sequencing technique.

The Next-Generation sequencing platform (technique) (referring to Metzker ML. Sequencing technologies—the next generation. Nat Rev Genet, 2010 January; 11(1):31-46, which is incorporated herein by reference) includes but not limited to Illumina-Solexa (GA™ HiSeg2000™, etc.), ABI-Solid and Roche-454 (Pyrosequencing) sequencing platform; the single molecule sequencing platform (technique) includes but not limited to True Single Molecule DNA sequencing technique of Helicos company, single molecule real-time (SMRT™) of Pacific Biosciences company, and nanopore sequencing technique of Oxford Nanopore Technologies company, etc. (referring to Rusk, Nicole (2009 Apr. 1). Cheap Third-Generation Sequencing. Nature Methods, 6 (4): 244-245, which is incorporated herein by reference).

With continuous development of sequencing technology, it would be appreciated by those skilled in the art that other sequencing methods and apparatuses may be also used for whole genome sequencing.

According to specific embodiments of the present disclosure, sequencing the sequencing-library is performed using at least one selected from Illumina-Solexa, ABI-Solid, Roche-454, and single-molecule sequencing apparatus. Next, the obtained sequencing result is processed to determine the sequencing data from a pre-determined region. The term “pre-determined region” used herein should be broadly understood, referring to any regions in the nucleic acid molecule comprising a site at which a pre-determined event may occur. For a SNP analysis, the term “pre-determined region” may refer to a region comprising a SNP site. For an aneuploidy analysis, the term “pre-determined region” may refer to a full length or a partial length of a chromosome to be analyzed, namely, all sequencing data from the chromosome are selected. A method of selecting the sequencing data from a pre-determined region among the sequencing result may not be subjected to any special restrictions, according to embodiments of the present disclosure, the sequencing data from the pre-determined region may be obtained by means of aligning the obtained sequencing result with a known nucleic acid reference sequence. In addition, prior to sequencing the sequencing-library, the method may further comprise a step of screening the sequencing-library, to directly obtain the sequencing data from the pre-determined region. Thus, according to embodiments of the present disclosure, a step of determining the sequencing data from the pre-determined region may be comprised after obtaining the sequencing data, by means of screening the sequencing result using an alignment method to obtain the sequencing data from the pre-determined region. Also, the sequencing result consisting of the sequencing data from the pre-determined region may be finally obtained, by means of selecting the sequencing-library prior to prior to sequencing the sequencing-library. According to embodiments of the present disclosure, a method of selecting the sequencing-library is not particularly limited, which may be performed at any step during the process of constructing the sequencing-library, for example, the sequencing-library may be selected using a probe specific for the pre-determined region. According to embodiments of the present disclosure, a genome may be fragmented to obtain a DNA fragment, the DNA fragment may be screened using a specific probe to obtain a screened DNA fragment, and a subsequent step of constructing a sequencing-library for the screened DNA fragment may be performed, to obtain the sequencing-library of the pre-determined region. Also, after the DNA sequencing-library is obtained, the sequencing-library may be screened using a probe specific for the pre-determined region, to obtain a screened sequencing-library of the pre-determined region. According to embodiments of the present disclosure, prior to sequencing the sequencing-library, the method may further comprise a step of screening the sequencing-library using a probe, which is specific for the pre-determined region. Thus, the step of preliminary screening the sequencing-library prior to the step of sequencing the sequencing-library, may raise a ratio between a data analyzable directly and all obtained sequencing data, and may further improve a sequencing depth, realize simultaneously sequencing and analyzing a plurality of pre-determined regions derived from a nucleic acid sample. According to embodiments of the present disclosure, a form of the probe is not particularly limited. According to embodiments of the present disclosure, the probe is provided in a chip. Thus, providing the probe on the chip may further improve the efficiency of analyzing the nucleic acid sample by means of realizing high-throughput screening the sequencing-library of a plurality of pre-determined regions, and may also further improve the efficiency of analyzing the nucleic acid sample. Those skilled in the art may design the probe according to the intended aim, and currently there are manufacturers providing probe synthesis and service of chip production, for example, a hybridization chip for MHC region may be designed, or a hybridization chip for a plurality of SNP (up to ten thousands orders of magnitude) may be designed. According to embodiments of the present disclosure, the method may comprise integrating a plurality of probes for the SNP site on a single chip, which may detect a plurality of diseases simultaneously by one hybridization reaction. Furthermore, the inventors find out that using the chip of detecting a monogenetic disease, the method according to the embodiments of the present disclosure, on the basis of being able to detect a large amount of the SNP sites, may realize an effective paternity test, and improve the validity and time-efficiency of the paternity test. And according to embodiments of the present disclosure, using the chip of detecting the monogenetic disease, the method according to the embodiments of the present disclosure, may detect an abnormality of a chromosome, for example, in an embodiment of the present disclosure, the method effectively realizes detecting a chromosome aneuploidy, such as Trisomy 21 syndrome. In addition, the method according to embodiments of the present disclosure may detect a plurality of samples simultaneously, by ligating different indexes having a known sequence during the process of constructing sequencing-library for each sample. The method according to embodiments of the present disclosure greatly improves the throughput of detection, reduces operation steps and reagent consumption of multiple tests in clinical application, saves time and reduces cost, which may provide a tremendous support for a large-scale application of clinical non-injured prenatal diagnosis in future.

In addition, according to embodiments of the present disclosure, a method of determining sequencing data from a pre-determined region by an alignment, may also combine with a method of screening a sequencing-library of the by a probe, to improve the precision of selecting the sequencing data from the pre-determined region. A detection of a pre-determined region having a relative shorter sequence, for example, for a detection aiming at determining a SNP mutation type, may screen sequencing data by screening sequencing-library only using a probe provided in a hybridization chip. In addition, according to embodiments of the present disclosure, the step of selecting sequencing data may further comprise removing sequencing data having a poor sequencing quality from the sequencing result, which may be filtered in accordance with a pre-determined standard by those skilled in the art. According to embodiments of the present disclosure, after obtaining the sequencing result, the method of detecting a pre-determined event in a nucleic acid sample may further comprise aligning the sequencing result with a known nucleic acid sequence, to obtain a uniquely aligned sequence; and selecting sequencing data from the pre-determined region among the uniquely aligned sequence. Thus, the accuracy or the efficiency of detecting and analyzing the nucleic acid sample may be further improved.

After selecting the sequencing data from the pre-determined region among the sequencing data, the method of detecting a pre-determined event in a nucleic acid sample may comprise determining an occurrence of the pre-determined event in the nucleic acid sample based on a composition of the sequencing data from the pre-determined region. For the sequencing data from the pre-determined region, particularly a sequencing result obtained by a high-throughput sequencing method using a Next-Generation sequencing platform, although a same site of the pre-determined region will be sequenced for several times, there will be a certain deviation or an occurrence of other mutations. The term “composition of the sequencing data” used herein refers to that, for the targeted region, all the sequencing data may comprise an obtained sequencing result of all sites, and the number of reads corresponding to various results. The inventors propose that the composition of these sequencing data may be analyzed using a statistical analyzing method, to exclude an accidental error and obtain a sequencing result which is most likely to reflect the truth.

For this purpose, inventors provide an analyzing method for the SNP site. For the analyzing method of the SNP site, the pre-determined region is a nucleic acid fragment comprising a known SNP, the pre-determined event is a mutation type of a SNP site, in which the step of determining an occurrence of the pre-determined event in the nucleic acid sample may further comprise determining a number ratio between the number of sequencing data with base A, T, G or C of the SNP site and the number of a total sequencing data respectively; and determining a base having a highest occurrence probability of the SNP site based on the number ratio by means of Bayesian Model, to determine the mutation type of the SNP site in the nucleic acid sample. Thus, the mutation type of the SNP site in the pre-determined region may be effectively determined, and then the paternity test may be performed by detecting a plurality of mutation types of the SNP site in fetus and parents thereof. And the analyzing method for the SNP site may be used to effectively detect a plurality of mutation types, which extends the scope of disease detection.

The inventors find out that at a specific site, the occurrences of four type bases (A, T, C and G) are mutually exclusive, and there are only four kinds of possibilities, as a result the occurrence probability of a specific base at the specific site follows a quadrinomial distribution. Thus, in the case of homozygote, such as AA, the occurrence probability of each base is shown as followings:

base

| | A | T | C | G |
|---|---|---|---|---|
| Pr(Base)* | 1 − δ | δ/3 | δ/3 | δ/3 |

Notes:
*Pr(Base) represents an occurrence probability of a base;
δ represents an error ratio of base, i.e. a ratio that a base is mis-sequenced during the sequencing process.

And in the case of heterozygous, such as AT, the occurrence probability of each base is shown as followings:

| base | A | T | C | G |
|---|---|---|---|---|
| Pr(Base)* | $\frac{1}{2} - \frac{δ}{3}$ | $\frac{1}{2} - \frac{δ}{3}$ | δ/3 | δ/3 |

Notes:
*Pr(Base) represents an occurrence probability of a base; δ represents an error ratio of base, i.e. a ratio that a base is mis-sequenced during the sequencing process.

According to the law of quadrinomial distribution, the occurrence probability is in the case of an occurring a_{A} times, T occurring a_{T} times, C occurring a_{C} times, G occurring a_{G} times among a sequencing result having the number of n:

$$Pr ( sequence \vert genotype = i ) = \frac{n !}{{a}_{A} ! {a}_{T} ! {a}_{C} ! {a}_{G} !} {p}_{A}^{{a}_{A}} {p}_{T}^{{a}_{T}} {p}_{C}^{{a}_{C}} {p}_{G}^{{a}_{G}} ,$$

in which a_{A}+a_{T}+a_{V}+a_{G=n},
PA, PT, pc and p_{G} represent the occurring probability of base A, T, C and G respectively, i∈{AA, TT, CC, GG, AT, AC, AG, CT, CG, GT} Since the sequencing depth of the current sequencing technology is relative high, there is no need to introduce a prior probability. As a result, prior to an observation, the occurrence probability of each genotype is assumed to be equal, i.e. Pr(genotype=i)=0.1, in which there are 10 kinds of occurrences in a sample space i∈{AA, TT, CC, GG, AT, AC, AG, CT, CG, GT}.

Based on the previous condition, the sequencing result may be analyzed by means of Bayesian Model, i.e. by means of the following equation:

$$Pr ( sequence = i \vert genotype ) = \frac{Pr ( genotype = i ) \cdot Pr ( sequence \vert genotype = i )}{{\sum }_{j} Pr ( genotype = j ) \cdot Pr ( sequence \vert genotype = j )} \\ i ∈ \{ AA , TT , CC , GG , AT , A C , AG , CT , CG , GT \} \qquad ( Formula I )$$

Formula I is the expansion of Bayesian Model, which may calculate a probability of an obtained sequencing result corresponding to different genotypes of a pre-determined region in a nucleic acid sample. A genotype having a maximum probability is the actual genotype determined by the analysis method according to embodiments of the present disclosure. Pr(genotype=i) refers to an occurrence probability of a certain kind of genotype, based on the previous analysis, the occurrence probabilities used herein are all defaulted as 0.1. Pr(sequence|genotype=i) represents a probability of an obtained sequencing data corresponding to actual genotype i, which may obtained by calculating with a formula:

$$Pr ( sequence \vert genotype = i ) = \frac{n !}{{a}_{A} ! {a}_{T} ! {a}_{C} ! {a}_{G} !} {p}_{A}^{{a}_{A}} {p}_{T}^{{a}_{T}} {p}_{C}^{{a}_{C}} {p}_{G}^{{a}_{G}} ;$$

in which Pr(genotype=i|sequence) represents an occurrence probability of different genotypes corresponding to the current sequencing data.

The analyzing method using Bayesian Model may calculate the occurrence probability of the specific base at the specific site among the sequencing result, to obtain a sequencing result having a maximum probability. Thus, the genotype for the specific site may be determined. Namely, the genotype having a highest occurrence probability may be determined as the genotype) at this specific site. In addition, Pr(genotype=i|sequencing) corresponding to an obtained genotype having a highest occurrence probability by sequencing may be converted to a quality value according to a formula −10*log₁₀ (Pr), which may evaluate the reliability of the genotype determination, in which Pr represents an occurrence probability of the genotype.

Thus, the method according to embodiments of the present disclosure may effectively determine the type of the specific site in the nucleic acid sample, for example, the method according to embodiments of the present disclosure may determine a plurality of mutation types of the SNP site simultaneously, thereby the method according to embodiments of the present disclosure may effectively detect consanguinity among the nucleic acid samples, realize an effective paternity test, and realize an effective detection for a plurality of disease simultaneously. It would be appreciated by those skilled in the art that the analysis method by means of Bayesian Model may be also suitable for analyzing a variation of other nucleic acids. Being different with traditional PCR method, the method of the present disclosure not only involves a plurality of sites, but also obtains a more reliable detection result, and can be used to detection a plurality of samples simultaneously which greatly increases the throughput and simplifies the operation procedure to a greater degree.

In addition, the present disclosure also provides a method of analyzing chromosome aneuploidy. According to an embodiment of the present disclosure, the pre-determined region is a first chromosome in genome; the pre-determined event is an aneuploidy of the first chromosome. According to another embodiment, determining an occurrence of the pre-determined event in the nucleic acid sample further comprises:

Firstly, a step of determining a number ratio between the number of sequencing data of the first chromosome and the number of the total sequencing data, which may determine the sequencing data of the first chromosome by aligning the sequencing data to a known genome information, and then the number ratio between the number of sequencing data of the first chromosome and the number of the total sequencing data may be obtained by comparison. The term “first chromosome” used herein should be understood broadly, which may refer to any target chromosome desired to be investigated, of which the number is not limited to one chromosome but even may be all chromosomes. According to embodiments of the present disclosure, the first chromosome is at least one selected from a group consisting of human chromosome 21, chromosome 18, chromosome 13, chromosome X and chromosome Y. Thus, the chromosomal disease common in human may be effectively determined. The inventors of the present disclosure surprisingly find out that, the method of determining the chromosome aneuploidy according to embodiments of the present disclosure may be effectively applied to detect aneuploidies of human chromosome 21, chromosome 18, chromosome 13, chromosome X and chromosome Y. Thus, the method of determining the chromosome aneuploidy according to embodiments of the present disclosure may be effectively applied to prenatal diagnosis, which may greatly shorten the detection time, avoid invasive injury to the pregnant women and reduce the miscarriage risk by conventional detection. According to embodiments of the present disclosure, a resource of the nucleic acid sample used in investigation of the chromosome aneuploidy is not subjected to special restriction, according to a specific embodiment of the present disclosure, the nucleic acid sample is a genomic DNA extracted from maternal plasma. Thus, on the premise of non-invasive injury to the fetus, the method of the present disclosure, may further realize detection of chromosome aneuploidy-related genetic disease with fetus. The noninvasive sampling method in the present disclosure avoids the miscarriage risk by conventional detection, such as an amniocentesis method, without using an ancillary device, such as an ultrasound method, which makes the sampling more simple and convenient.

Next, after obtaining the number ratio between the number of sequencing data of the first chromosome and the number of the total sequencing data, if the aneuploidy exists, there will be a significant difference of the number ratio between the number of sequencing data of the first chromosome and the number of the total sequencing data with a normal nucleic acid sample. Thus, based on the difference of the number ratio between the number of sequencing data of the first chromosome and the number of the total sequencing data with a preset parameter, whether the nucleic acid sample has an aneuploidy of the first chromosome can be determined. Then, the effective determination of chromosome aneuploidy may result in an effective detection of fetal genetic disease in prenatal diagnosis. The term “preset parameter” used herein refers to a relevant data regarding a certain chromosome obtained by subjecting a nucleic acid sample with a genome known to be normal to repeating the protocol and analysis conducted to a single cell of a biological sample. It would be appreciated by those skilled in the art that a relevant parameter of a certain chromosome and a relevant parameter of a chromosome from a normal nucleic acid sample may be obtained using a same condition for sequencing and a same mathematics method, respectively. Here, the relevant parameter of the chromosome from the normal nucleic acid sample may be taken as a control reference. In addition, the term “preset” used herein should be understood broadly, which may be determined by an experiment in advance, or may be obtained from a parallel experiment when performing analysis with the biological sample. Thus, according to an embodiment of the present disclosure, the preset parameter is a number ratio between the number of sequencing data of the first chromosome and the number of the total sequencing data thereof from the normal nucleic acid sample. According to embodiments of the present disclosure, the difference of the number ratio between the number of sequencing data of the first chromosome and the number of the total sequencing data with the preset parameter may be expressed using any known mathematical method, for example, the number ratio may be compared with the present parameter, and then the obtained result may be compared with a threshold, if the obtained result is greater than the threshold, the nucleic acid sample is determined to be trisomy of the first chromosome. In addition, according to an embodiment of the present disclosure, the method may further comprise calculating the number ratio and the preset parameter using student's t test, which may further improve the accuracy and precision of the analysis result with the sequencing data. It would be appreciated by those skilled in the art that, after performing relevant statistical test, an analysis method similar with the above analysis may be performed by setting different threshold accordingly. According to embodiments of the present disclosure, after performing student's t test, the threshold may be set to at least 1.5, for example at least 2, more preferably at least 3.

System for Detecting a Pre-Determined Event in a Nucleic Acid Sample

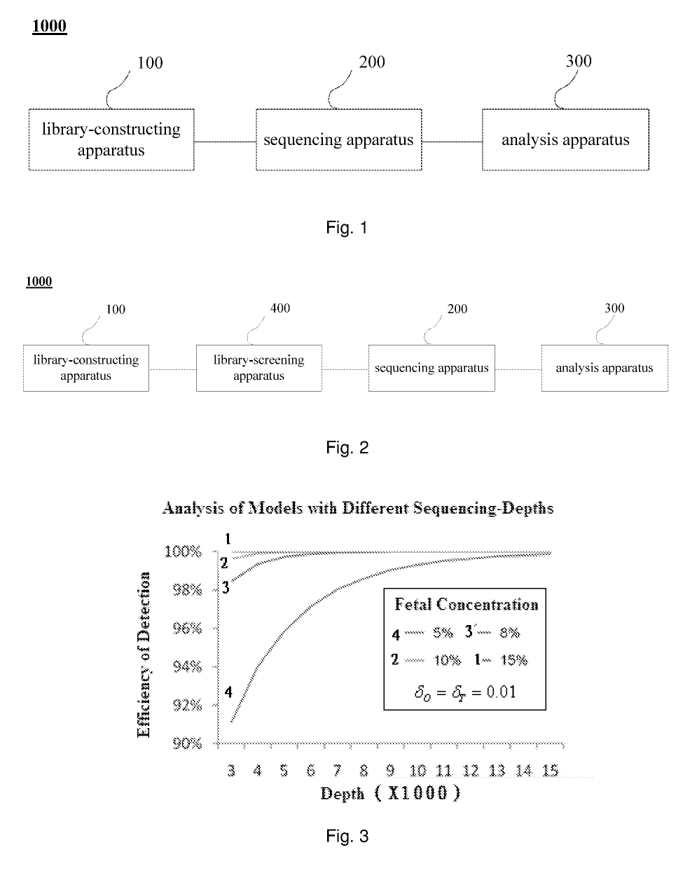

According to a second broad aspect of the present disclosure, there is provided a system 1000 for detecting a pre-determined event in a nucleic acid sample. Referring to FIG. 1, according to embodiments of the present disclosure, the system 1000 for detecting the pre-determined event in the nucleic acid sample may comprise a library-constructing apparatus 100, a sequencing apparatus 200, and an analysis apparatus 300. The system 1000 for detecting the pre-determined event in the nucleic acid sample according to embodiments of the present disclosure may effectively carry out the method of detecting the pre-determined event in the nucleic acid sample according to embodiments of the present disclosure. The advantages of the method have been described in detail previously, so a detailed description thereof will be omitted here.

According to embodiments of the present disclosure, the library-constructing apparatus 100 is suitable for constructing a sequencing-library for a nucleic acid sample. According to embodiments of the present disclosure, the method and the process of constructing the sequencing-library for the nucleic acid sample may be selected appropriately by those skilled in the art according to different sequencing techniques. A detailed process may refer to a specification provided by sequencing-instrument manufacturer, such as Illumina Company, for example Multiplexing Sample Preparation Guide (Part#1005361; February 2010) or Paired-End SamplePrep Guide (Part#1005063; February 2010) is referred, which are both incorporated herein by reference. According to embodiments of the present disclosure, a method and an apparatus for extracting a nucleic acid sample from a biological sample are also not subjected to any special restrictions, which may be a commercial kit for nucleic acid extraction.

According to embodiments of the present disclosure, the sequencing apparatus is connected to the library-constructing apparatus, and is suitably for sequencing the sequencing-library to obtain a sequencing result consisting of a plurality of sequencing data. According to embodiments of the present disclosure, a method and an apparatus for performing sequencing are not subjected to any special restrictions. According to embodiments of the present disclosure, the sequencing apparatus may be a Next-Generation sequencing technique, also may be a Third-Generation and a Fourth-Generation or a more advanced sequencing technique. According to specific embodiments of the present disclosure, the whole genome sequencing-library may be sequenced by at least one selected from Illumina-Solexa, ABI-Solid, Roche-454, and single-molecule sequencing apparatus. Thus, combining with a latest sequencing technique, the sequencing depth for one single site may achieve a deeper extent, and the sensitivity and the accuracy of detection may be greatly improved, thus, the efficiencies of the detection and analysis for the nucleic acid sample may be further improved by utilizing these sequencing apparatuses having characteristics of high-throughput and deep sequencing. Thus, the precision and accuracy of subsequent analysis with the sequencing data may be further improved, particularly when performing statistic testing analysis. Referring to FIG. 2, according to an embodiment of the present disclosure, the system may further comprise a library-screening apparatus 400. According to an embodiment of the present disclosure, the library-screening apparatus 400 is configured with a probe specific for the pre-determined region, to screen the sequencing-library by using the probe. Thus, the sequencing-library may be preliminary screened before sequencing step, thereby the number ratio which can be directly subjected to analysis among the obtained sequencing data may be increased, and the sequencing depth may further improved, realizing performing the sequencing and analysis with a plurality of pre-determined regions in the nucleic acid sample simultaneously. According to an embodiment of the present disclosure, the probe is provided in a chip. Thus, by realizing screening the sequencing-library of a plurality of pre-determined regions by configuring the probe in the chip, the method may further improve the efficiency of detecting and analyzing the nucleic acid sample. As stated above, the library-screening apparatus 400 described herein may be configured in any steps of the library construction, for example, the library-screening apparatus 400 may be configured after breaking the nucleic acid sample (e.g. genome DNA) to obtain the DNA fragment, and also may be configured after obtaining the sequencing-library of genome DNA, and before performing sequencing step.

According to embodiments of the present disclosure, the analysis apparatus 300 is connected to the sequencing apparatus 200, and is suitable for receiving the sequencing data from the sequencing apparatus 200, selecting the sequencing data from the pre-determined region among the sequencing data, and further determining the occurrence of the pre-determined event based on the number of the sequencing data from the pre-determined region. Selection the sequencing data from the determined region among the sequencing data has been described in detail previously, so a detailed description thereof will be omitted here. According to embodiments of the present disclosure, relevant sequence information may be pre-stored in the analysis apparatus 300, and the analysis apparatus 300 also may be connected to a remote database (not shown in figures) performing operation online.

The determination regarding the occurrence of the pre-determined event has been described in detail previously, so a detailed description thereof will be omitted here. In short, the analysis apparatus 300 is suitable for SNP detection and analysis. For the method of SNP analysis, the pre-determined region is a nucleic acid fragment comprising a known SNP site, the pre-determined event is a mutation type of a SNP site. Specifically, the analysis apparatus 300 is suitable for: determining a number ratio between the number of sequencing data with base A, T, G or C of the SNP site and the number of a total sequencing data respectively; and determining a base having a highest occurrence probability of the SNP site based on the number ratio by means of Bayesian Model, to determine the mutation type at the SNP site in the nucleic acid sample. Thus, the method of SNP analysis according to embodiments of the present disclosure may effectively determine the mutation type at the SNP site in the pre-determined region, and then the paternity test may be performed by detecting the mutation type at a plurality of the SNP sites with fetus and parents thereof.

According to an embodiment of the present disclosure, the analysis apparatus 300 may be used in analyzing a chromosome aneuploidy, in which the pre-determined region is a first chromosome in a genome, and the pre-determined event is an aneuploidy of the first chromosome, specifically, the analysis apparatus 300 is suitable for: determining a number ratio between the number of sequencing data of the first chromosome and a number of the total sequencing data; and determining whether the nucleic acid sample has the aneuploidy with respect to the first chromosome, based on a difference between the number ratio and a preset parameter. Thus, the chromosome aneuploidy may be effectively determined, which may realize an effective detection of fetal genetic disease in prenatal diagnosis. According to an embodiment of the present disclosure, the first chromosome is at least one selected from a group consisting of human chromosome 21, chromosome 18, chromosome 13, chromosome X and chromosome Y. Thus, the chromosomal disease being common in human may be effectively determined. According to an embodiment of the present disclosure, the analysis apparatus may further comprise a statistical testing apparatus (not shown in figures), to perform student's t test with the number ratio and the preset parameter. Thus, the accuracy and precision of the analysis result with the sequencing data may be further improved.

Using the system of detecting a pre-determined event in a nucleic acid sample, may effectively implement the method of detecting the pre-determined event in the nucleic acid sample above-described, for example, may effectively detect a mutation type of the SNP sites, or may effectively analyze chromosomal aneuploidy prenatally. The term “connect” used herein should be understood broadly, which may be a direct connection, or an indirect connection, as long as the connection of the above functions can be achieved.

It should be noted that it would be appreciated by those skilled in the art that the characteristics and the advantages of the method for detecting the pre-determined event in the nucleic acid sample described above are also suitable for the system for detecting the pre-determined event in the nucleic acid sample, so a detailed description thereof will be omitted here.

Capturing Chip

According to a third broad aspect of the present disclosure, there is provided a capturing chip used in the method of detecting a pre-determined event in a nucleic acid sample described previously. Referring to FIG. 5, the capturing chip 2000 may comprise: a capturing chip body 2001 and a plurality of oligonucleotide probes 2002. According to embodiments of the present disclosure, the plurality of oligonucleotide probes 2002 are configured on a surface of the capturing chip body 2001, in which, the plurality of oligonucleotide probes are specific for the pre-determined region of human genome. Thus, by utilizing the capturing chip, the pre-determined region of the nucleic acid sample may be effectively captured among the nucleic acid sample, which may effectively improve the efficiency of the method for detecting the pre-determined event in the nucleic acid sample. According to embodiments of the present disclosure, firstly an interested pre-determined region is determined, and then the oligonucleotide sequence is determined in accordance with characteristic of sequence in the pre-determined region. According to embodiments of the present disclosure, a type of the pre-determined region is not subjected to special restrictions. According to embodiments of the present disclosure, the pre-determined region is a gene region relating to a disease in human genome. Thus, by utilizing the chip, disease-related gene information can be screened out from a human genome. According to specific embodiments of the present disclosure, the gene region locates at chromosome 18, chromosome 13 or chromosome 21 in human genome. In addition, according to embodiments of the present disclosure, the pre-determined region is a nucleic acid fragment comprising a known SNP site. Thus, utilizing the chip may screen out a large amount of SNP-related information simultaneously.

It should be noted that it would be appreciated by those skilled in the art that the characteristics and the advantages of the method for detecting the pre-determined event in the nucleic acid sample described above are also suitable for the capturing chip, so a detailed description thereof will be omitted here.

### Method of Monogenic Disorder Diagnosis, Paternity Test or Prenatal Screening Down's Syndrome

According to a fourth broad aspect of the present disclosure, there is provided a method of monogenic disorder diagnosis, paternity test or prenatal screening Down's syndrome. According to embodiments of the present disclosure, the method may comprise: detecting a pre-determined event in a nucleic acid sample according to the method of detecting a pre-determined event in a nucleic acid sample above-mentioned.

Reference will be made in detail to examples of the present disclosure. It should be noted that the following examples are explanatory, and cannot be construed to limit the scope of the present disclosure.

If not specified, the used techniques in the examples are conventional methods well-known to people skilled in the art, which may be performed in accordance with Molecular Cloning (3rd Ed.) or relevant products, and all reagents and products used in the example are also commercially available. Various processes and methods without detailed description are all known conventional methods, the resource, trade name and components need to be explicated are all indicated when appearing first time, and all the same reagents thereafter are identical with the previous with the indication unless a special statement.

### Example 1 Detection of SNP Site

The samples comprising a maternity peripheral blood and a peripheral blood from the father of the same family, and a fetal cord blood after the birth were collected in centrifuge tubes having EDTA anticoagulation, respectively. A centrifuge tube containing the maternity peripheral blood sample was centrifuged at 1600 g for 10 minutes at 4° C. to separate blood cell and plasma. The separated plasma was then centrifuged at 1600 g for 10 minutes at 4° C. again, to further remove residual leukocytes. The blood cell and the plasma separated from the maternity peripheral blood were subjected to DNA extraction using TIANamp Micro DNA Kit (TIANGEN), respectively, which represented a maternal genome DNA and a genome DNA mixture of maternity and fetus. The other two samples of the peripheral blood from the father of the same family and the fetal cord blood were all subjected to DNA extraction using the above kit. The obtained all DNA samples, except for a DNA sample extracted from the plasma, should be subjected to fragmenting using Covaris™ instrument, to obtain a DNA fragment having a size of 500 bp. The obtained DNA fragment was then subjected to library construction in accordance with the specification provided by the manufacturer of HiSeq2000™ sequencer from Illumina Company, to obtain a sequencing library. A specific process was shown as follows:

End-Repairing

| | | |
|---|---|---|
| 10x polynucleotide kinase buffer | 10 | μL |
| dNTPs (10 mM) | 4 | μL |
| T4 DNA polymerase | 5 | μL |
| Klenow fragment (having an activity of 5′→3′ | 1 | μL |
| polymerase and an activity of 3′→5′ exonuclease) | | |
| T4 polynucleotide kinase | 5 | μL |
| DNA | 30 | μL |
| ddH₂O | up to 100 | μL |

The tube containing the above system was allowed reaction for 30 minutes at 20° C., and then the end-repaired product was purified using a PCR purification kit (QIAGEN). Then, the purified sample was dissolved in 34 μL of the elution buffer.

Adding Base a to the End-Repaired DNA at 3′-End

| | |
|---|---|
| 10x Klenow buffer | 5 μL |
| dATP (1 mM) | 10 μL |
| Klenow fragment (3′-5′ exo-) | 3 μL |
| DNA (the end-repaired DNA) | 32 μL |

The tube containing the above system was allowed reaction for 30 minutes at 37° C., and then the end-repaired DNA added with base A was purified using a MinElute® PCR purification kit (QIAGEN). Then, the purified sample was dissolved in 12 μL of the elution buffer.

Ligating an Adaptor

| | |
|---|---|
| 2x quick ligating buffer | 25 μL |
| PEI Adapter oligomix (20 μM) | 10 μL |
| T4 DNA ligase | 5 μL |
| obtained DNA added with base A at 3′-end | 10 μL |

The tube containing the above system was allowed reaction for 15 minutes at 20° C., and then the obtained DNA ligated to an adaptor was purified using a PCR purification kit (QIAGEN), and recycled. Then, the purified sample was dissolved in 32 μL of the elution buffer.

PCR Amplification:

| | |
|---|---|
| obtained DNA ligated to an adaptor | 10 μL |
| Phusion DNA polymerase Mix | 25 μL |
| PCR primer (10 pmol/μL) | 1 μL |
| Index N* (10 pmol/μL) | 1 μL |
| ddH₂O | 13 μL |

Note:
*provided by Illumina manufacturer

Procedure of PCR reaction was shown as follows:

| | | | |
|---|---|---|---|
| 98° C. | 30 s | | |
| 98° C. | 10 s | | |
| 65° C. | 30 s | {close oversize brace} | 10 cycles |
| 72° C. | 30 s | | |
| 72° C. | 5 min | | |
| 4° C. | Hold | | |

Then, the obtained amplification product was purified using a PCR Purification Kit (QIAGEN), and recycled. The purified and recycled product was finally dissolved in 50 μL of the elution buffer.

The constructed library was subjected to Agilent® Bioanalyzer 2100 to detect whether the distribution of the fragments met the requirement, and then the qualified library was subjected to quantification using Q-PCR method. After quantification, the qualified library was subjected to hybridization on a solid-phase chip 110321_HG19_BGI_exon_chrM_cap_HX3 customized by NimbleGen Company (details of the chip were shown below). The hybridized product was sequenced using Illumina® HiSeg2000™ sequencer, the number of sequencing cycle was PE101Index (i.e. dual 101 bp index sequencing). The parameter setting and operation method of the apparatus were performed in accordance with the operating specification of HiSeg2000™ sequencer provided by manufacturer of Illumina® Company (the operating specification may be obtained from the Illumina® Company website.

The design and preparation of solid-phase chip 110321_HG19_BGI_exon_chrM_cap_HX3:

According to design guidance for probe provided by manufacture of Roche NimbleGen, aiming at the regions listed in the following table, selecting the monogenic disease-related regions, taking a known human genome sequence Hg19 as a reference sequence, the inventors designed 7644 probes having an average length of 150 bp, of which the coverage is 1.8 M of the region in the reference sequence. The information of the probe designing was submitted to Roche NimbleGen Company to synthesis in a hybridization chip, namely, 110321_HG19_BGI_exon_chrM_cap_HX3. As an alternative, probe design also may be completed by a chip company, as long as the region effectively covered by the probe can achieve a same or a similar effect.

target region

| chromosome | start | end |
|---|---|---|
| chr1 | 6400000 | 217600000 |
| chr2 | 26600000 | 228200000 |
| chr3 | 33000000 | 191200000 |
| chr4 | 900000 | 178400000 |
| chr5 | 68700000 | 169600000 |
| chr6 | 33100000 | 155700000 |
| chr7 | 6000000 | 143100000 |
| chr8 | 24800000 | 119200000 |
| chr9 | 34600000 | 140100000 |
| chr10 | 26200000 | 123400000 |
| chr11 | 2100000 | 121100000 |
| chr12 | 48300000 | 103400000 |
| chr13 | 20700000 | 78500000 |
| chr14 | 21100000 | 88500000 |
| chr15 | 34500000 | 91400000 |
| chr16 | 1400000 | 53800000 |
| chr17 | 3500000 | 79900000 |
| chr18 | 21100000 | 44300000 |
| chr19 | 1200000 | 50900000 |
| chr20 | 56900000 | 58000000 |
| chr21 | 33000000 | 45200000 |
| chr22 | 18500000 | 51100000 |
| chrX | 7100000 | 154300000 |

The amount of the obtained sequencing data was shown as table-1. The sequencing depths of the leukocyte samples of parents and fetus were about 50×, the sequencing depth of maternity peripheral blood sample was about 300×. During the process of data analysis, sequencing reads were aligned to the reference sequence hg19 using SOAP v2.20, with a setting parameter (-v 5-s 40-1 40-r 1). In the alignment results, only those sequencing reads which can be uniquely aligned to the target region of the chip were subjected to subsequent analysis. For SNP result of parents and fetus, data of the existing whole genome sequencing and the chip was taken as a standard result. Thus, all SNP sits locating at the target region of the chip were selected therefrom as a candidate site for analysis.

TABLE 1
the amount of sequencing data

| | | | | | | | specificity of |
|---|---|---|---|---|---|---|---|
| | target | data | the number | | coverage | average | capture |
| sample | region | (M) | of reads | length | (%) | depth | (%) |
| father | 1,797,207 | 64.93 | 728,226 | 100 | 97.45 | 36.13 | 60.74 |
| mother | 1,797,207 | 93.29 | 1,043,992 | 100 | 97.97 | 51.91 | 61.47 |
| fetus | 1,797,207 | 596.00 | 6,782,558 | 100 | 99.46 | 331.63 | 6.54 |
| after the | | | | | | | |
| birth | | | | | | | |

A coverage and distributions of A, T, G, C at each SNP site were calculated, those sites having relative low coverage were filtered, a base distribution of an inferable site was finally obtained. A determination of genotype in parents' genome and a determination of fetal genotype in maternity peripheral blood according to Bayesian Model shown as formula I, was shown in table-2 with specific data.

TABLE 2
Calculation of SNP accuracy

| Sample | | total | average depth | accurate | Percentage |
|---|---|---|---|---|---|
| father | | 765 | 78.8 | 765 | 100% |
| mother | | 639 | 57.7 | 638 | 99.84% |
| fetus | mother | 67 | 412.0 | 62 | 92.54% |
| | homozygote | | | | |
| | mother | 35 | 370.3 | 11 | 31.43% |
| | heterozygote | | | | |
| | total | 102 | 397.7 | 73 | 71.57% |

As shown in table 2, the accuracy of genotype detection for parents was substantially 100%, the accuracy of fetal genotype detection was also 70% or more, in which the accuracy of site detection corresponding to mother homozygote may achieve 92.54%, the accuracy was not high resulted from mother heterozygous site. At the present, the result is restricted by sequencing depth of the current experiment. As shown in FIG. 3, an analysis result with simulated data indicated that the accuracy can be further greatly improved as increasing the sequencing depth. FIG. 3 was an accuracy result of different sequencing depths obtained by calculating stimulation frequency of each bases using by means of Bayesian Model shown as formula I, in which the stimulation frequencies are corresponding to different sequencing depths randomly produced, according to a probability distribution of bases in the case of mother heterozygote and fetus homozygote.

### Example 2 Detection of a Chromosome Aneuploidy

One maternity plasma sample, which had been determined as a Trisomy 21 (Trisomy 21 syndrome) with fetus by a detection result using amniocentesis, and two plasma samples of maternities having normal fetus were selected. The above three samples were subjected to DNA extraction, then the obtained DNA samples were subjected to library-constructing in accordance with the method shown in example 1. The obtained sequencing-library was subjected to sequencing capture using a capturing chip being same with example 1, the captured library was sequenced using Illumina HiSeq2000™ sequencer. For abnormality detection of chromosome number, the effective data obtained from sequencing were shown in Table-3. The sequencing depth of each sample was about 50×.

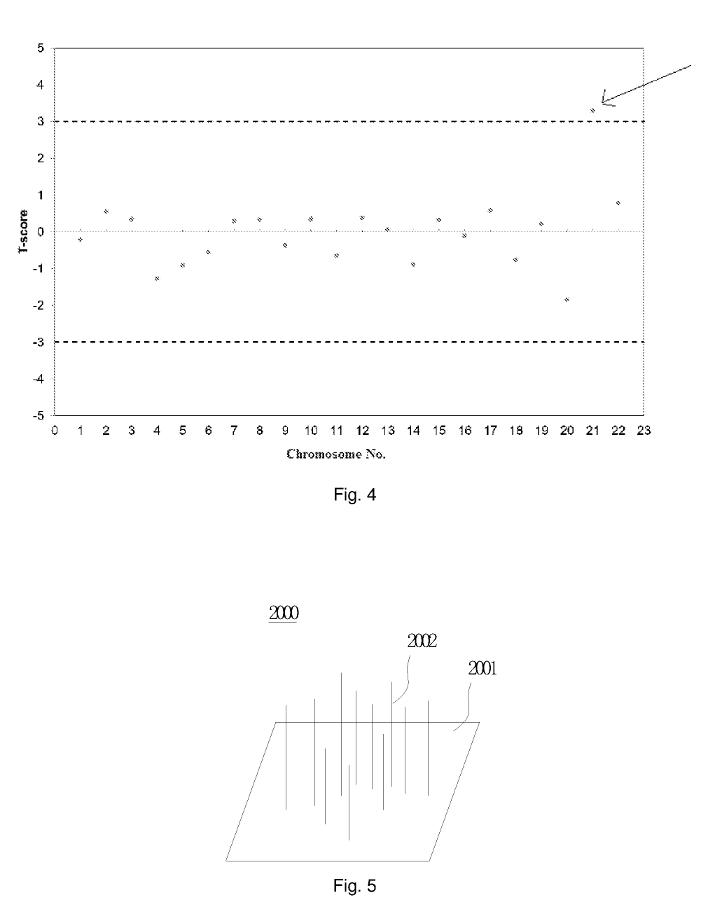

The alignment process was conformed with SNP genotype determination in example 1. For alignment result, a number ratio of the number of reads uniquely aligned to each chromosome and the number of sequencing data with a whole genome was calculated. Then a ratio from a normal sample taken as a control was subjected to deduction, and the obtained relative reads distribution was subjected to a student's t test, in which those having an outliers exceeded the significant limitations were determined as a chromosome having an abnormal number. As shown in FIG. 4, for T21 plasma sample, the statistical results of all other chromosomes were all within the threshold, while the statistical result of chromosome 21 exceeded the threshold (3), shown as an arrow in FIG. 4. The number abnormality of chromosome 21 may be successfully detected by threshold screening.

TABLE 3
The amount of sequencing data

| | | | | | | | specificity |
|---|---|---|---|---|---|---|---|
| | target | data | the number | | coverage | average | of capture |
| sample | region | (M) | of reads | length | (%) | depth | (%) |
| control 1 | 1,797,207 | 596.00 | 6,782,558 | 100 | 99.46 | 331.63 | 6.54 |
| control 2 | 1,797,207 | 50.05 | 572,255 | 100 | 99.35 | 27.85 | 59.54 |
| T21 | 1,797,207 | 43.44 | 496,024 | 100 | 99.26 | 24.17 | 58.25 |

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

What is claimed is:

1. A method of detecting a pre-determined event in a nucleic acid sample comprising:
constructing a sequencing-library for the nucleic acid sample;
sequencing the sequencing-library to obtain a sequencing result consisting of a plurality of sequencing data;
determining the sequencing data from a pre-determined region; and
determining an occurrence of the pre-determined event in the nucleic acid sample based on a composition of the sequencing data from the pre-determined region.

2. The method of claim 1, wherein the pre-determined region is a nucleic acid fragment comprising a known SNP, the pre-determined event is a mutation type of a SNP site,
wherein
determining an occurrence of the pre-determined event in the nucleic acid sample further comprises:
determining a number ratio between the number of sequencing data with base A, T, G or C of the SNP site and the number of a total sequencing data respectively; and
determining a base having a highest occurrence probability of the SNP site based on the number ratio by means of Bayesian Model, to determine the mutation type of the SNP site in the nucleic acid sample.

3. The method of claim 1, wherein the pre-determined region is a first chromosome in a genome, the pre-determined event is an aneuploidy of the first chromosome, wherein determining an occurrence of the pre-determined event in the nucleic acid sample further comprises:
determining a number ratio between the number of sequencing data of the first chromosome and the number of the total sequencing data; and
determining whether the nucleic acid sample has the aneuploidy with respect to the first chromosome, based on a difference between the number ratio and a preset parameter.

4. A system for detecting a pre-determined event in a nucleic acid sample comprising:
a library-constructing apparatus, suitable for constructing a sequencing-library for the nucleic acid sample;
a sequencing apparatus, connected to the library-constructing apparatus, suitable for sequencing the sequencing-library to obtain a sequencing result consisting of a plurality of sequencing data;
an analysis apparatus, suitable for determining the sequencing data from a pre-determined region and determining an occurrence of the pre-determined event in the nucleic acid sample based on a composition of the sequencing data from the pre-determined region.

5. The system of claim 4, wherein the pre-determined region comprises a nucleic acid fragment comprising a known SNP, the pre-determined event is a mutation type of a SNP site,
wherein the analysis apparatus is suitable for:
determining a number ratio between the number of sequencing data with base A, T, G or C of the SNP site and the number of a total sequencing data respectively; and
determining a base having a highest occurrence probability of the SNP site based on the number ratio by means of Bayesian Model, to determine the mutation type of the SNP site in the nucleic acid sample.

6. The system of claim 4, wherein the pre-determined region is a first chromosome in a genome, the pre-determined event is an aneuploidy of the first chromosome,
wherein
the analysis apparatus is for:
determining a number ratio between the number of sequencing data of the first chromosome and a number of the total sequencing data; and
determining whether the nucleic acid sample has the aneuploidy with respect to the first chromosome, based on a difference between the number ratio and a preset parameter.

7. The method of claim 1, the nucleic acid sample is at least one selected from a group consisting of hum an genomic DNA sample and free nucleic acid.

8. The method of claim 7, the genomic DNA sample is a genomic DNA derived from human white blood cell or maternal plasma.

9. The method of claim 1, sequencing the sequencing-library is performed using at least one selected from Illumina-Solexa, ABI-Solid, Roche-454, and single-molecule sequencing apparatus.

10. The method of claim 1, prior to sequencing the sequencing-library, wherein the method further comprises a step of screening the sequencing-library using a probe, wherein the probe is specific for the pre-determined region.

11. The method of claim 10, the probe is provided in a chip.

12. The method of claim 1, after obtaining the sequencing result, wherein the method further comprises:
aligning the sequencing result with a known nucleic acid sequence, to obtain a uniquely aligned sequence; and
selecting the sequencing data from the pre-determined region among the uniquely aligned sequence.

13. The method of claim 3, wherein the first chromosome is at least one selected from a group consisting of human chromosome 21, chromosome 18, chromosome 13, chromosome X and chromosome Y.

14. The method of claim 3, wherein the nucleic acid sample is a genomic DNA extracted from maternal plasma.

15. The method of claim 3, wherein the preset parameter is a number ratio between the number of sequencing data of the first chromosome and the number of the total sequencing data thereof, wherein the number ratio is obtained from a normal human nucleic acid sample.

16. The method of claim 3, wherein the method further comprises calculating the number ratio and the preset parameter using student's t test.

17. The system of claim 4, wherein the sequencing apparatus is at least one selected from Illumina-Solexa, ABI-Solid, Roche-454, and single-molecule sequencing apparatus.

18. The system of claim 4, wherein the system further comprises a library-screening apparatus configured with a probe specific for the pre-determined region, to screen the sequencing-library by using the probe.

19. The system of claim 6, wherein the first chromosome is at least one selected from a group consisting of human chromosome 21, chromosome 18, chromosome 13, chromosome X and chromosome Y.

20. The system of claim 6, the analysis apparatus further comprises a student t-statistic test apparatus, for calculating the number ratio and the preset parameter using student t-statistic test.
